(19) United States
(12) Patent Application Publication
(10) Pub. No.: US 2012/0157964 A1
(43) Pub. Date: Jun. 21, 2012

(54) DEVICE AND METHOD FOR THE DELIVERY OF MEDICINAL LIQUID DIRECTLY FROM A SMALL BOTTLE (VIAL)

(76) Inventor: Shlomo Uri HAIMI, Or Akiva (IL)

(21) Appl. No.: 12/973,941

(22) Filed: Dec. 21, 2010

(51) Int. Cl. A61M 5/20; A61M 35/00

(52) U.S. Cl. 604/506; 604/146; 604/310

(57) ABSTRACT

A device for the delivery of medicinal liquids, in which the device includes a base configured for sealing attachment to a container pre-filled with liquid medicine and a displaceable actuator element associated with the base. The association is that of a piston and a cylinder such that the base and the displaceable actuator element define between them a pressure generating volume. The base includes a piercing element having at least one air delivery passage providing fluid communication between the pressure generating volume and an interior of the container, the piercing element also includes at least one liquid delivery passage providing fluid communication between the interior of the container and a medicinal delivery element.

### FIELD AND BACKGROUND OF THE INVENTION

The present invention relates to devices for the delivery of medicinal liquids from a storage vial to a patient and, in particular, it concerns a device for the delivery of medicinal liquids directly from a storage vial to a patient.

The most common manner of storing liquid medicines is in what are referred to as standard vials. Such vials are closed with at lid includes a septum and the liquid medicine is removed using a standard syringe and needle.

Due to the drawbacks associated with this manner of drug delivery, a number of attempts have been made to provide liquid medicine delivery devices which are easier and safer to use.

One such device is disclosed in U.S. Pat. No. 7,470,258 to Barker et al. The Barker device is configured for use with a standard medicine vial which is attached to the device. Air is forced into the vial in order to deliver the liquid medicine in the vial to a transfer chamber in the device. Upon further manipulation, the liquid medicine is then forced out of the transfer chamber and injected into the patient.

Drawbacks to the Barker device include being a complex mechanism that is expensive to produce. Further, the use of the transfer chamber provides an opportunity to contaminate the liquid medicine.

There is therefore a need for a device for the delivery of liquid medicines that easy and safe to use and simple and inexpensive to produce and delivers the liquid medicine directly from the storage vial to the patient.

### SUMMARY OF THE INVENTION

The present invention is a device for the delivery of medicinal liquids directly from a storage vial to a patient.

According to the teachings of the present invention there is provided, [TO BE COPIED IN FROM CLAIMS WHEN FINALIZED]

### BRIEF DESCRIPTION OF THE DRAWINGS

The invention is herein described, by way of example only, with reference to the accompanying drawings, wherein:

FIG. 1 is a cross section of a first preferred embodiment of a device for the delivery of medicinal liquids directly from a storage vial to a patient constructed and operational according to the teachings of the present invention, shown here before delivery of the liquid medicine;

FIG. 2 is a cross section of the device of FIG. 1, shown after delivery of the liquid medicine;

FIG. 3 is a cross section of a second preferred embodiment of a device for the delivery of medicinal liquids directly from a storage vial to a patient constructed and operational according to the teachings of the present invention, shown here before delivery of the liquid medicine; and

FIG. 4 is a cross section of a third preferred embodiment of a device for the delivery of medicinal liquids directly from a storage vial to a patient constructed and operational according to the teachings of the present invention, shown here before delivery of the liquid medicine.

### DESCRIPTION OF THE PREFERRED EMBODIMENTS

The present invention is a device for the delivery of medicinal liquids directly from a storage vial to a patient.

The principles and operation of a device for the delivery of medicinal liquids according to the present invention may be better understood with reference to the drawings and the accompanying description.

By way of introduction, the liquid delivery device of the present invention removes liquid from a vial by inserting air into the vial, thereby creating positive pressure within the vial that forces the liquid out of the vial. It will be appreciated that since the interior volume of the vial is open to ambient pressure through a liquid delivery passage, when air is forced into the interior volume of the vial no compression of the air or the liquid occurs. Therefore, the volume of air forced into the interior volume of the vial determines the volume of the liquid medicine delivered to the medicinal delivery element, and therefore, the volume of liquid medicine delivered to the patient. With this in mind, it will be understood that the liquid delivery device of the present invention may be configured such that the volume of air forced into the interior volume of the vial is predetermined based on the desired dose of the liquid medicine required. The volume of air forced into the interior volume of the vial may be predetermined by providing a variety of liquid delivery device each having a different predetermined delivery volume. Such predetermined delivery volume may force out only a portion of the liquid in the vial, or it may be adequate to completely empty the vial. Alternatively, the pressure generating volume of the delivery device of the present invention may itself be variable, allowing the user to determine the dose to be delivered.

It should be noted that although the drawings depict the delivery device of the present invention used in conjunction with a medical vial and therefore the term vial is used throughout the description, this is not intended as a limitation and the delivery device of the present invention may be configured for attachment to substantially any container such as, but not limited to, bottles and jars. Therefore, the term “vial” as it is used herein is intended to include substantially any suitable container, therefore, the terms “vial” and “container” are used interchangeably herein. Nor should the term “medicine” used herein be considered a limitation, but rather as an example of a type of liquid suitable for used in the embodiments shown in the drawings.

As will be discussed below, the delivery device of the present invention may be configured with any one of a number of different medicinal delivery elements, thereby facilitating different ways of administering the liquid to a patient. Therefore, administration may be as a subcutaneous injection with a needle; a nasal spray; an ear spray; an eye spray; an oral spray; a spray to the surface of the skin; and rectal administration.

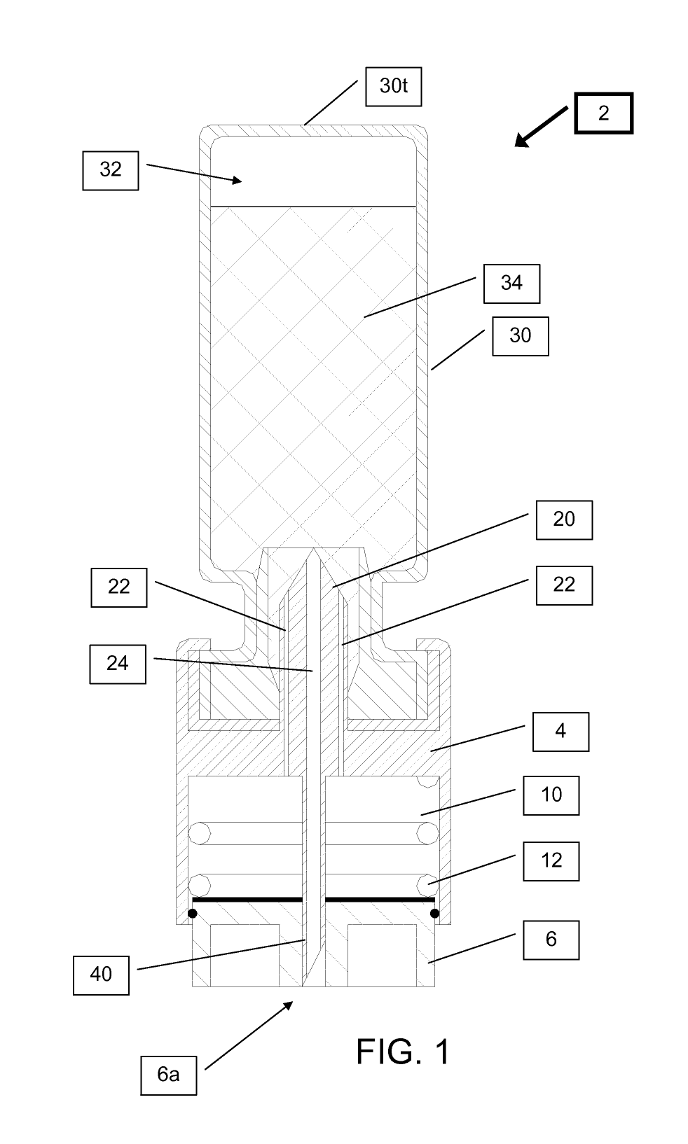

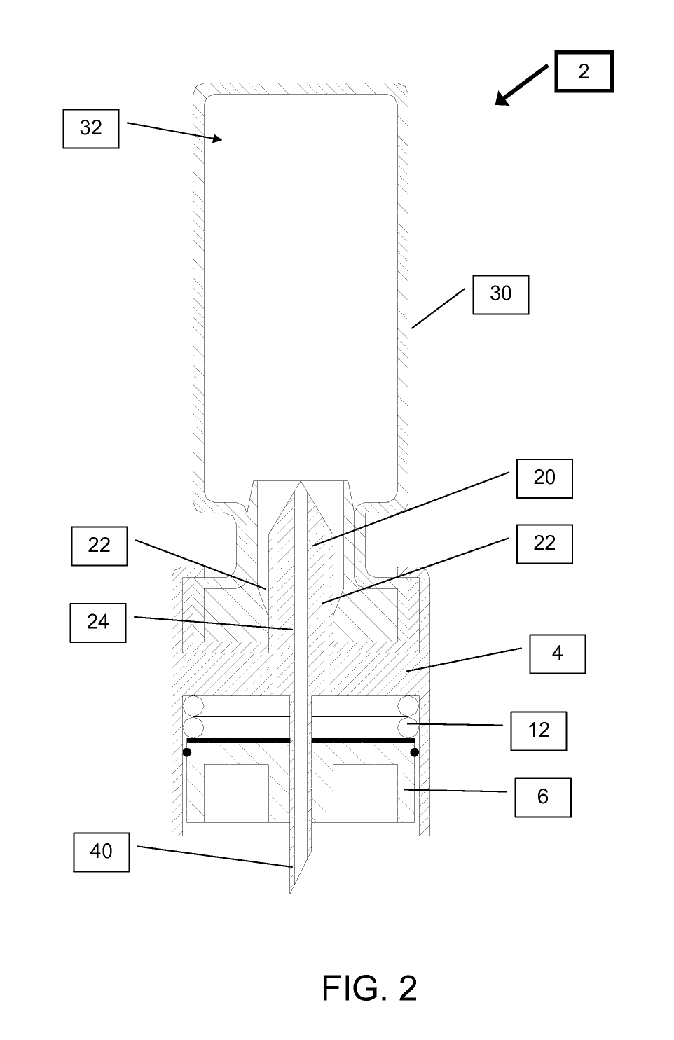

Referring now to the drawings, FIGS. 1 and 2 illustrate a first preferred embodiment of the liquid delivery device 2 of the present invention. In this embodiment, base 4 includes an open region that is configured to accept displaceable actuator element 6. The association of base 4 and displaceable actuator element 6 is that of a cylinder (base 4) and piston (displaceable actuator element 6) so as to define between them a pressure generating volume 10.

While the circumferential contour of the walls of pressure generating volume 10 and displaceable actuator element 6 may be of substantially any suitable closed curve.

A biasing element, such as coil spring 12 maintains displaceable actuator element 6 in a storage (non-delivery) position prior to use.

Base 4 also includes a piercing element 20, which is so referred to herein due to its ability to pierce the septum of a standard medical vial. It should be noted, however, that the function of this element of the present invention is the same in all embodiments of the delivery device of the present invention regardless of the need to pierce a septum or not.

The piercing element 20 has at least one air delivery passage 22 providing fluid communication between the pressure generating volume 10 and an interior 32 of the container 30, illustrated here, by way of example only, as a standard medical vial. The piercing element 20 also includes at least one liquid delivery passage 24 providing fluid communication between the interior 32 of the container 30 and a medicinal delivery element 40, illustrated here, by way of example only, as an injection needle.

Displacement of displaceable actuator element 6 within the pressure generating volume 10 forces air from the pressure generating volume 10 through the air delivery passage 22 and into the interior 32 of vial 30, thereby forcing the liquid medicine 34 from the vial 30 through liquid delivery passage 24 to the medicinal delivery element 40.

As illustrated in FIG. 2, displacement of displaceable actuator element 6 into the pressure generating volume 10 exposes at least a portion of the medicinal delivery element 40. Therefore, operation of this embodiment of the liquid delivery device 2 of the present invention includes attaching vial 30 to the base 4. Placing face 6a of displaceable actuator element 6 against the skin of the patient in an area suitable for the required treatment, in this case, by example only, a subcutaneous injection with a needle. Pressure is then applied to the top 30t of the vial in a direction toward the patient so as to force the displaceable actuator element 6 into the pressure generating volume 10, while simultaneously forcing the injection needle into the target tissue for delivery of the medicinal liquid 34 in the vial 30 as described above. It will be appreciated that upon removal of the liquid delivery device 2 from the patient, spring 12 returns displaceable actuator element 6 to its original position in which the injection needle (medicinal delivery element 40) is safely hidden.

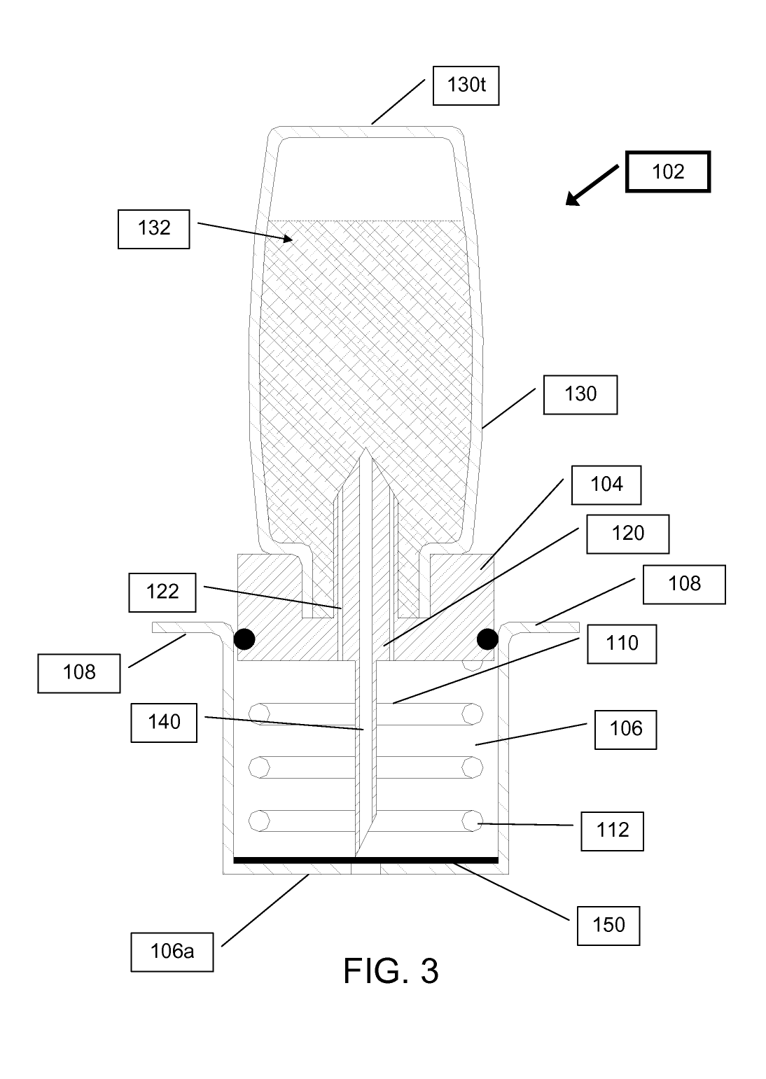

FIG. 3 illustrates a second preferred embodiment of the liquid delivery device 102 of the present invention. In this embodiment, displaceable actuator element 106 includes an open region that is configured to accept base 104. Here too, the association of base 104 and displaceable actuator element 106 is that of a cylinder (displaceable actuator element 106) and piston (base 104) so as to define between them a pressure generating volume 110. It will be understood that the structure and operation of the piercing element 120 and medicinal delivery element 140 is the same as their respective counterparts described above.

This second embodiment of the liquid delivery device 102 of the present invention resembles a traditional syringe in that displaceable actuator element 106 is supported by the fingers of the user against extensions 108 and pressure is allied by the user's thumb to the top 130t of vial 130 in order to operate the device and deliver the liquid medicine 132 to the patient.

Deployed adjacent to the end wall 106a of displaceable actuator element 106 is a septum 150 that provides a pressure resistant seal around the injection needle (medicinal delivery element 140) as the base 104 is displaced into the pressure generating volume 110 configured in the displaceable actuator element 106.

Here too, after use, spring 112 returns the displaceable actuator element 106 to its original position in which the injection needle (medicinal delivery element 140) is safely hidden.

It will be appreciated that in the embodiments of FIGS. 1, 2 and 3 the longitudinal axis of vial 130 and the longitudinal axis of the medicinal delivery element 140 are substantially parallel. This is well suited for delivery of liquid medication when the delivery device 2, 102 is in an upright (vertical) position. However, problems arise when attempting to deliver liquid with when the delivery device 2, 102 is in a horizontal position.

To answer this problem, the third preferred embodiment of the liquid delivery device 200 of the present invention. This embodiment includes a base 204 that maintains the vertical deployment of the vial 230 while providing a medicinal delivery element 140 that is deployed in a horizontal position. That is to say the longitudinal axis of vial 230 and a longitudinal axis of the medicinal delivery element 240 are perpendicular to each other.

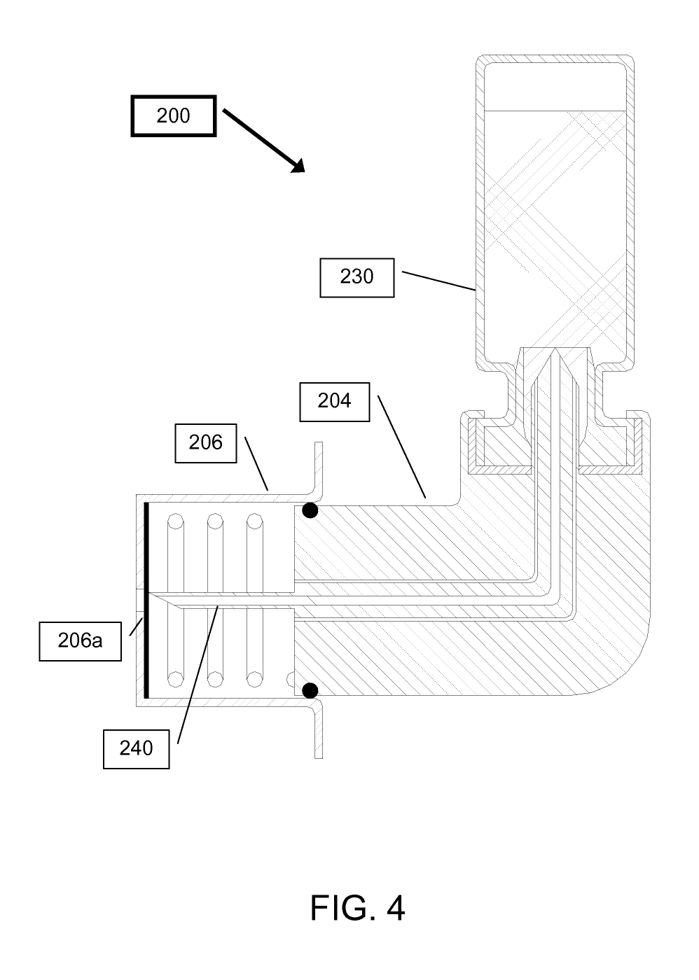

While the embodiments of FIGS. 3 and 4 are shown herein with the medicinal delivery element 240 configured as an injection needle, this configuration of the cylinder and piston association of the base 204 and displaceable actuator element 206 is well suited for other medicinal delivery elements such as discussed above.

It will be appreciated that a number of additional features may be included in liquid delivery device of the present invention, among them are:
Adaptors can be produced for a wide variety of vials.
Stoppers that can prevent unwanted discharge of the contents of the vial.
Injection prior to the needle's penetration into the body can be prevented.
Innervating/confusing the nerves by adding unevenness to the area of liquid delivery device that comes into contact with the skin in order to eliminate or at least drastically reduce the pain of the injection.
Prevention of air being injected at the end of the injection of the liquid.
Ensuring injection of the entire contents of the small bottle.
Indication of the end of an injection.
The ability to inject partial doses with the aid of scale marks.
Proof of injection after the action is carried out.
Tamper evidence, with a mechanic element and/or sterile packaging that is torn before use.
Suction can be carried out prior to the injection for injection into the muscle.

It will be appreciated that the above descriptions are intended only to serve as examples and that many other embodiments are possible within the spirit and the scope of the present invention.

What is claimed is:

1. A device for the delivery of medicinal liquids, the device comprising:
(a) a base configured for sealing attachment to a container, said container being pre-filled with liquid medicine; and
(b) a displaceable actuator element associated with said base, said association being that of a piston and a cylinder such that said base and said displaceable actuator element define between them a pressure generating volume;
wherein said base includes a piercing element having at least one air delivery passage providing fluid communication between said pressure generating volume and an interior of said container, said piercing element also including at least one liquid delivery passage providing fluid communication between said interior of said container and a medicinal delivery element.

2. The device of claim 1, wherein said base is configured as said cylinder and said displaceable actuator element is configured as said piston such that said displaceable actuator element is displaceable within said base.

3. The device of claim 1, wherein said base is configured as said piston and said displaceable actuator element is configured as said cylinder such that said displaceable actuator element is displaceable around said base.

4. The device of claim 1, wherein said vial is a standard medicine vial.

5. The device of claim 1, wherein said pressure generating volume has a predetermined capacity so as to establish a predetermine dosage of said liquid medicine to be delivered.

6. The device of claim 1, wherein said a medicinal delivery element is one from a list that includes an injection needle, a spray head, a rectal insertion head.

7. The device of claim 6, wherein said spray head is configured to spray liquid medicine to one from a list that includes mouth, nose, ear, eye and surface of the skin.

8. The device of claim 1 wherein a longitudinal axis of said vial and a longitudinal axis of said medicinal delivery element are parallel.

9. The device of claim 1 wherein a longitudinal axis of said vial and a longitudinal axis of said medicinal delivery element are perpendicular.

10. A method for the delivery of medicinal liquids to a patient, the method comprising:
(a) providing a medicinal delivery device having:
(i) a base configured for sealing attachment to a container;
(ii) a displaceable actuator element associated with said base, said association being that of a piston and a cylinder such that said base and said displaceable actuator element define between them a pressure generating volume;
wherein said base includes a piercing element having at least one air delivery passage providing fluid communication between said pressure generating volume and an interior of said container, said piercing element also including at least one liquid delivery passage providing fluid communication between said interior of said container and a medicinal delivery element.
(b) attaching a container pre-filled with liquid medicine to said base;
(c) displacing said displaceable actuator element so as to force air from said pressure generating volume through said air delivery passage and into said vial, thereby forcing said liquid medicine from said vial through liquid delivery passage to said medicinal delivery element; and
(d) delivering said liquid to the patient.

11. The method of claim 10, wherein said step of displacing said displaceable actuator element includes displacing said displaceable actuator element within said base.

12. The method of claim 10, wherein said step of displacing said displaceable actuator element includes displacing said displaceable actuator element around said base.

13. The method of claim of claim 10, wherein said step of displacing said displaceable actuator element further includes deploying said medicinal delivery element for use.

14. The method of claim 13, wherein said deploying said medicinal delivery element for use includes inserting an injection needle into bodily tissue of the patient.
